(19) United States
(12) Patent Application Publication
(10) Pub. No.: US 2017/0143716 A1
(43) Pub. Date: May 25, 2017

(54) METHOD OF TREATING PROLIFERATIVE DISORDERS AND OTHER PATHOLOGICAL CONDITIONS MEDIATED BY BCR-ABL, C-KIT, DDR1, DDR2 OR PDGF-R KINASE ACTIVITY

(71) Applicant: Novartis AG, Basel (CH)

(72) Inventors: Tomasz Szczudlo, New York, NY (US); Richard Woodman, Annandale, NJ (US); Ophelia Yin, Denville, NJ (US)

(21) Appl. No.: 15/425,417

(22) Filed: Feb. 6, 2017

Related U.S. Application Data
(63) Continuation of application No. 14/797,716, filed on Jul. 13, 2015.
Application No. 14/797,716 is a division of application No. 14/264,357, filed on Apr. 29, 2014, now abandoned.
Application No. 14/264,357 is a continuation of application No. 13/501,274, filed on Apr. 11, 2012, now abandoned.
(60) Provisional application No. 61/254,323, filed on Oct. 23, 2009.

(51) Int. Cl. A61K 31/506

(52) U.S. Cl. CPC A61K 31/506

(57) ABSTRACT

The present invention relates to a regimen for the administration of a pyrimidyl-aminobenzamide of formula 1 as defined herein for the treatment of proliferative disorders, particularly solid and liquid tumors, and other pathological conditions mediated by the Bcr-Abl oncoprotein, the cell transmembrane tyrosine kinase receptor c-Kit, DDR1 (discoidin domain receptor 1), DDR2 (discoidin domain receptor 2) or PDGF-R (platelet derived growth factor receptor) kinase activity.

The present invention relates to a regimen for the administration of a pyrimidylaminobenzamide of formula I

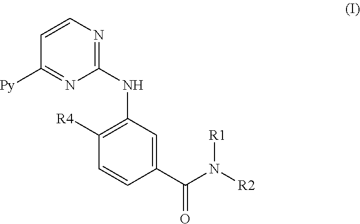

wherein
(a) Py denotes 3-pyridyl,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and
R₃ represents hydroxy, lower alkoxy, acyloxy, carboxy, lower alkoxycarbonyl, carbamoyl, N- mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
or
(b) Py denotes 5-pyrimidyl, R₁ is hydrogen, R₂ is [[(3S)-3-(dimethylamino)- 1-pyrrolidinyl]methyl]-3-(trifluoromethyl)phenyl and R₄ is methyl;
or of a pharmaceutically acceptable salt thereof,
for the treatment of proliferative disorders, particularly solid and liquid tumors, and other pathological conditions mediated by the Bcr-Abl oncoprotein, the cell transmembrane tyrosine kinase receptor c-Kit, DDR₁ (discoidin domain receptor 1), DDR₂ (discoidin domain receptor 2) or PDGF-R (platelet derived growth factor receptor) kinase activity.

### BRIEF DESCRIPTION OF THE DRAWINGS

FIG. 1 depicts the mean concentration of nilotinib in Patients treated with 400 mg nilotinib twice daily.

FIG. 2 depicts the hypothesis that QHS dosing is associated with increased bioavailability of nilotinib.

The compound of formula I, wherein Py denotes 3-pyridyl, R₁ represents hydrogen, R₂ represents 5-(4-methyl-1H-imidazol-1-yl)-3-(trifluoromethyl)-phenyl and R₄ represents methyl, is known under the International Non-proprietary Name “nilotinib”. Nilotinib (4-methyl-3-[[4-(3-pyridinyl)-2-pyrimidinyl]amino]-N-[5-(4-methyl-1H-imidazol-1-yl)-3-(trifluoromethyl)phenyl] benzamide) is approved and marketed in the form of its monohydrochloride monohydrate salt under the brand name Tasigna™. Nilotinib is an ATP-competitive inhibitor for Bcr-Abl and also inhibits c-Kit, DDR_{1,} DDR₂ and PDGF-R kinase activity at clinically relevant concentrations. Tasigna™ is available as 200 mg hard capsule for oral administration for the treatment of Philadelphia-positive chronic myeloid leukaemia (CML) in the chronic phase (CP) and accelerated phase (AP) in patients resistant to or intolerant of at least one prior therapy including imatinib. For the treatment of CML a daily dose of 800 mg of nilotinib is applied in two doses of 400 mg each.

The effect of food on the pharmacokinetic parameters of 400 mg oral dose of nilotinib in the formulation mentioned above was studied in human subjects. The concomitant administration of nilotinib with food significantly increased subjects exposure, especially in high fat meals. In said study the total exposure (AUC₀₋ₜ) was 82% and Cₘₐₓ was 112% after a high fat breakfast, whereas the increase in total exposure (AUC₀₋ₜ) was 29% and Cₘₐₓ was 55% after a light breakfast given 30 minutes prior to dosing. In view of these findings, it is recommended that nilotinib shall not be taken with a meal in order to minimize the effect of food on nilotinib bioavailability. A statement in this regard is, for instance, included in sections 4.2, 4.4 and 4.5 of the SPC (Summary of Product Characteristics) of the marketing authorization for Tasigna™ issued by the European Medicines Agency (EMEA).

The present invention is based on the conclusion that once daily bedtime dosing (QHS) of nilotinib is associated with a systemic exposure comparable to that of the current used dosing of 300 mg BID, so that the total daily dose of drug products comprising nilotinib can be reduced compared to the dose required under the same medical circumstances when using a conventional treatment regimen.

In a study in healthy volunteers as described in the Examples, a slight diurnal effect on nilotinib pharmacokinetics (PK) was confirmed. Nilotinib exposure was shown to be up to 20% higher following the evening dose than the morning dose.

Further, it was found that when a pyrimidylaminobenzamide of formula I is administered to a human once daily QHS the risk of food drug interaction is minimized. The instant treatment regimen provides patients with a convenient once daily dosing, thus improving patient compliance. The instant treatment regimen offers the benefit of maintaining efficacy of the pyrimidylaminobenzamide of formula I while reducing the food effect observed when using a conventional treatment regimen.

Hence, the present invention relates to the use of pyrimidylaminobenzamides of formula I

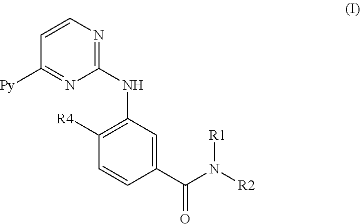

wherein the radicals have the meanings as provided above, or of a pharmaceutically acceptable salt thereof alone or in combination with other active compounds for the preparation of a medicament for the treatment of proliferative disorders and other pathological conditions mediated by Bcr-Abl, c-Kit, DDR_{1,} DDR₂ or PDGF-R kinase activity, wherein the medicament is adjusted in manner to be used once daily at bedtime (QHS).

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tent-butyl, propyl, such as n-propyl or isopropyl, ethyl or methyl. Preferably lower alkyl is methyl, propyl or tert-butyl.

Lower acyl is preferably formyl or lower alkylcarbonyl, in particular acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihydroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g. by methyl, by halogen-lower alkyl, e.g. trifluoromethyl, or by phenyl; lower alkylene dioxy bound to two adjacent C-atoms, e.g. methylenedioxy, lower alkyl, e.g. methyl or propyl; halogen-lower alkyl, e.g. trifluoromethyl; hydroxy-lower alkyl, e.g. hydroxymethyl or 2-hydroxy-2-propyl; lower alkoxy-lower alkyl; e.g. methoxymethyl or 2-methoxyethyl; lower alkoxycarbonyl-lower alkyl, e.g. methoxycarbonylmethyl; lower alkynyl, such as 1-propynyl; esterified carboxy, especially lower alkoxycarbonyl, e.g. methoxycarbonyl, n-propoxy carbonyl or iso-propoxy carbonyl; N-mono-substituted carbamoyl, in particular carbamoyl monosubstituted by lower alkyl, e.g. methyl, n-propyl or iso-propyl; amino; lower alkylamino, e.g. methylamino; di-lower alkylamino, e.g. dimethylamino or diethylamino; lower alkylene-amino, e.g. pyrrolidino or piperidino; lower oxaalkylene-amino, e.g. morpholino, lower azaalkylene-amino, e.g. piperazino, acylamino, e.g. acetylamino or benzoylamino; lower alkylsulfonyl, e.g. methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substitutents selected from the group defined above as substitutents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g. pyrrolidino, 2-oxopyrrolidino or piperidino; lower oxaalkylene-amino, e.g. morpholino, or lower azaalkylene-amino, e.g. piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic heteroaryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g. acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula I and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substitutents selected from the group defined above as substitutents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)2-one. In another preferred embodiment, the pyrimidinyl radical is substituted by hydroxy both in position 2 and 4 and hence exists in several tautomeric forms, e.g. as pyrimidine-(1H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g. 2- or 3-morpholinyl, 2-oxo-1H-1-azepin-3-yl, 2-tetrahydrofuranyl, or 2-methyl-1,3-dioxolan-2-yl.

Pyrimidylaminobenzamides within the scope of formula I, wherein py is 3-pyridyl and the process for their manufacture are disclosed in WO 04/005281, which is hereby incorporated into the present application by reference.

The pyrimidylaminobenzamide of formula I wherein Py denotes 5-pyrimidyl, R₁ is hydrogen, R₂ is [[(3S)-3-(dimethylamino)-1-pyrrolidinyl]methyl]-3-(trifluoromethyl)phenyl and R₄ is methyl is also known as INNO-406. The compound, its manufacture and pharmaceutical compositions suitable for its administration are disclosed in EP1533304A.

Pharmaceutically acceptable salts of pyrimidylaminobenzamides of formula I, wherein py is 3-pyridyl, are especially those disclosed in WO2007/015871. In one preferred embodiment nilotinib is employed in the form of its monohydrochloride monohydrate. WO2007/015870 discloses certain polymorphs of nilotinib and pharmaceutically acceptable salts thereof useful for the present invention. A suitable formulation for the administration of nilotinib monohydrochloride monohydrate is described in WO2008/037716.

As used herein, the expression “proliferative disorders, particularly solid and liquid tumors, and other pathological conditions mediated by the Bcr-Abl oncoprotein, the cell transmembrane tyrosine kinase receptor c-Kit, DDR₁ (discoidin domain receptor 1), DDR₂ (discoidin domain receptor 2) or PDGF-R (platelet derived growth factor receptor) kinase” activity means melanoma, especially melanoma harboring c-KIT mutations, breast cancer, cancer of the colon, lung cancer, cancer of the prostate or Kaposi's sarcoma, gastrointestinal stromal tumors (GIST), acute myeloid leukemia (AML), leukemia which responds to an inhibition of the Abl tyrosine kinase activity, such as chronic myeloid leukemia (CML) and Philadelphia chromosome positive acute lymphoblastic leukemia (Ph+ ALL), mesothelioma, systemic mastocytosis, hypereosinophilic syndrome (HES), fibrosis, especially hepatic fibrosis and renal fibrosis, rheumatoid arthritis, polyarthritis, scleroderma, lupus erythematosus, graft-versus host diseases, neurofibromatosis, pulmonary hypertension, especially, pulmonary arterial hypertension, Alzheimer's disease, seminomas and dysgerminomas and psoriasis. Preferably, the regime described herein is applied in the following disorders and conditions: GIST, CML, Ph+ ALL, systemic mastocytosis, HES, fibrosis, scleroderma, neurofibromatosis, pulmonary arterial hypertension.

In one embodiment of the present invention the disorder is selected from CML and Ph+ ALL, more preferably CML.

In another embodiment of the present invention the disorder is selected from GIST and melanoma, especially melanoma harboring c-KIT mutations.

In another embodiment of the present invention the disorder is selected from systemic mastocytosis and HES.

In a further embodiment of the present invention the disorder is selected from systemic scleroderma, neurofibromatosis and pulmonary arterial hypertension.

As used herein, the expression “Cₘₐₓ” means maximum peak concentration in plasma.

As used herein, the expression “AUC” means area under the plasma concentration curve.

As used herein, the expression “QHS” means that the drug product containg a compound of formula (I) is taken by the human subjects just before bedtime, preferably evening bedtime. Importantly, the subject is not permitted to take any food at least for the last two hours before taking the drug product. The term “bedtime” implies that the subject is taking the drug product before resting or, preferably, sleeping for 3 to 12 hours, preferably 5 to 10 hours, more preferably between 6 and 8 hours. Sleeping can be night time sleep (preferred) or sleep any time during the day.

For the purposes of the present invention, nilotinib can be applied in a total daily dose of 400 to 1000 mg depending, in particular on the disease to be treated and the disease status of the patient under treatment.

In a further aspect of the invention, the treatment regimen described herein allows to lower the total daily dose applied to patients suffering from Philadelphia positive leukemia, especially CML CP, to 500 to 700 mg/day, especially to 600 mg/day. A lower dose is reducing the incidence of side effects correlating with the total drug load.

The present inventions also provides a method of treating or preventing proliferative disorders and other pathological conditions mediated by the Bcr-Abl oncoprotein, the cell transmembrane tyrosine kinase receptor c-Kit, DDR₁ (discoidin domain receptor 1), DDR₂ (discoidin domain receptor 2) or PDGF-R (platelet derived growth factor receptor) kinase activity in a subject in need thereof comprising administering a pyrimidylaminobenzamide derivatives of formula (I):

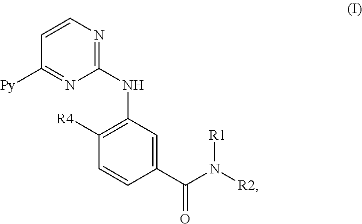

wherein
(a) Py denotes 3-pyridyl,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising 0-, 1-, 2- or 3-ring nitrogen atoms and 0 or 1 oxygen atom and 0 or 1 sulfur atom, which groups in each case are unsubstituted or mono- or poly-substituted; and
R₃ represents hydroxy, lower alkoxy, acyloxy, carboxy, lower alkoxycarbonyl, carbamoyl, N-mono- or N,N-di-substituted carbamoyl, amino, mono- or di-substituted amino, cycloalkyl, heterocyclyl, an aryl group, or a mono- or bi-cyclic heteroaryl group comprising 0-, 1-, 2- or 3-ring nitrogen atoms and 0 or 1 oxygen atom and 0 or 1 sulfur atom, which groups in each case are unsubstituted or mono- or poly-substituted; or
R₁ and R_{2,} together, represent alkylene with 4, 5 or 6 carbon atoms optionally mono- or di-substituted by lower alkyl, cycloalkyl, heterocyclyl, phenyl, hydroxy, lower alkoxy, amino, mono- or di-substituted amino, oxo, pyridyl, pyrazinyl or pyrimidinyl; benzalkylene with 4 or 5 carbon atoms; oxaalkylene with 1 oxygen and 3 or 4 carbon atoms; or azaalkylene with 1 nitrogen and 3 or 4 carbon atoms, wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-di-substituted carbamoyl-lower alkyl, cycloalkyl, lower alkoxycarbonyl, carboxy, phenyl, substituted phenyl, pyridinyl, pyrimidinyl or pyrazinyl;
R₄ represents hydrogen, lower alkyl or halogen;
or
(b) Py denotes 5-pyrimidyl, R₁ is hydrogen, R₂ is [[(3S)-3-(dimethylamino)- 1-pyrrolidinyl]methyl]-3-(trifluoromethyl)phenyl and R₄ is methyl;
or a pharmaceutically acceptable salt of such a compound, wherein the compound of formula I is administered once daily, preferably in the evening, just before bedtime.

In a preferred embodiment of the invention, the subject is not permitted to take any food at least for the last two hours before taking the drug product.

### EXAMPLES

### Example 1

### Study in CML Patients Obtaining 400 mg Nilotinib Twice Daily

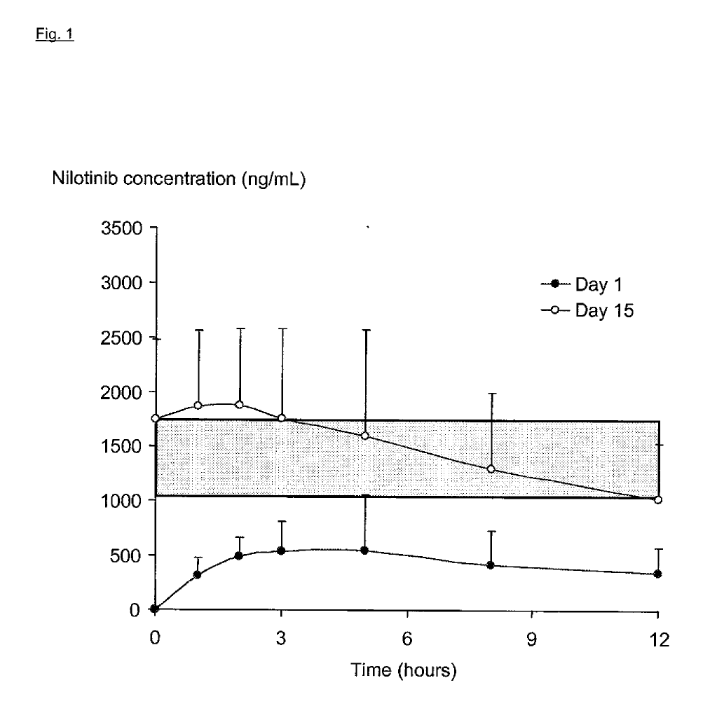

21 Patients were treated with 400 mg nilotinib twice daily. The mean concentration over time is shown in FIG. 1. Blood samples were collected prior to morning dose (C0) and prior to evening dose (C12). It was found that the ratio C0/C12 is 1.7. With other words, the trough concentration of nilotinib in the morning was 60 to 80% higher than that observed in the evening.

### Example 2

### Simulation of 600 mg QHS vs. 400 mg Twice Daily

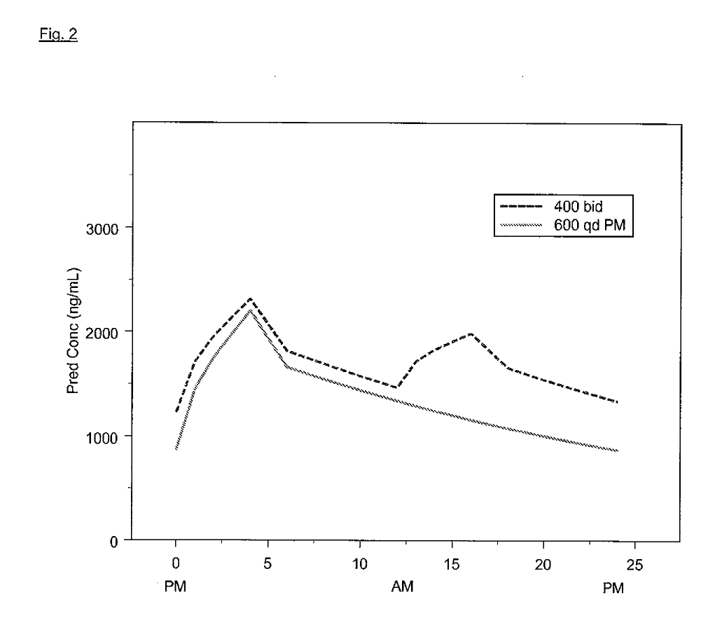

The simulation depicted in FIG. 2 is based on the hypothesis that QHS dosing is associated with increased bioavailability of nilotinib. Based on that assumption, Cₘₐₓ appears to be similar for both treatment approaches.

### Example 3

### PK Study in Healthy Volunteers

The increased exposure with QHS was confirmed in a study investigating nilotinib pharmacokinetics in healthy volunteers (HV) comparing cohorts receiving 600 mg morning dose or 600 mg morning dose QHS, respectively. In a single center, 4-way crossover study (n=16-24), HV group A was administered 300 mg nilotinib (in the form of nilotinib monohydrochloride monohydrate) in the morning, 2 hours after breakfast; HV group B was administered 300 mg nilotinib (in the form of nilotinib monohydrochloride monohydrate) in the evening, 2 hours after diner; HV group C was administered 600 mg nilotinib (in the form of nilotinib monohydrochloride monohydrate) in the evening, 2 hours after diner; and HV group D was administered 600 mg nilotinib (in the form of nilotinib monohydrochloride monohydrate) in the evening, 4 hours after diner.

TABLE 1
Study Results - Summary of PK Parameters

| | | | | | | | | | Geometric Mean Ratio | |
|---|---|---|---|---|---|---|---|---|---|---|
| | A | | B | | C | | D | | (90% Cls) | |
| Parameter | (N = 20) | | (N = 18) | | (N = 22) | | (N = 22) | | B vs A | D vs C |
| tₘₐₓ (h) | 4.0 | (3.0, 8.0) | 4.0 | (3.0, 10.0) | 4.0 | (3.0, 10.0) | 4.0 | (2.0, 10.2) | 0.49 (−1.00, | −0.49 (−5.96, |
| | | | | | | | | | 6.00) | 7.00) |
| Cₘₐₓ | 577 | (35) | 655 | (18) | 854 | (29) | 782 | (46) | 1.14 (1.01, | 0.92 (0.82, |
| (ng/mL) | | | | | | | | | 1.27) | 1.02) |
| AUC₀₋ₜₗₐₛₜ | 13650 | (27) | 15556 | (18) | 20819 | (22) | 19591 | (30) | 1.14 (1.06, | 0.94 (0.88, |
| (ng · h/mL) | | | | | | | | | 1.23) | 1.01) |
| AUC_{0-inf} | 14920 | (31) | 16272 | (19) | 23216 | (21) | 21937 | (34) | 1.09 (1.00, | 0.94 (0.87, |
| (ng · h/mL) | | | | | | | | | 1.19) | 1.03) |
| AUC0-12 | 4577 | (33) | 5537 | (16) | 7124 | (29) | 6650 | (41) | 1.21 (1.09, | 0.93 (0.85, |
| (ng · h/mL) | | | | | | | | | 1.34) | 1.03) |
| AUC0-24 | 7781 | (30) | 9435 | (18) | 11857 | (26) | 111064 | (36) | 1.21 (1.11, | 0.93 (0.86, |
| (ng · h/mL) | | | | | | | | | 1.32) | 1.01) |
| t_{1/2} (h) | 20.3 | (38) | 14.5 | (21) | 20.5 | (39) | 19.9 | (38) | NA | NA |

The nilotinib PK was compared when administered in the evening versus administration in the morning (B vs. A) and the potential residual food effect on nilotinib absorption was assessed (D vs. C).

### Example 4

### Phase III Study in CML Patients

The benefits described herein can be confirmed in a randomized phase III study in patients with newly diagnosed CML CP comparing 300 mg nilotinib twice daily with 600 mg QHS.

We claim:

1. A method of treating or preventing proliferative disorders and other pathological conditions mediated by the Bcr-Abl oncoprotein, the cell transmembrane tyrosine kinase receptor c-Kit, DDR₁ (discoidin domain receptor 1), DDR₂ (discoidin domain receptor 2) or PDGF-R (platelet derived growth factor receptor) kinase activity comprising administering a pyrimidylaminobenzamide derivatives of formula (I): 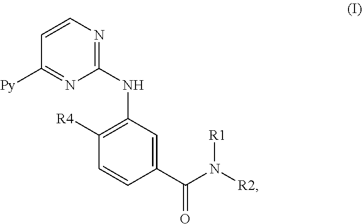
wherein
(a) Py denotes 3-pyridyl,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising 0-, 1-, 2- or 3-ring nitrogen atoms and 0 or 1 oxygen atom and 0 or 1 sulfur atom, which groups in each case are unsubstituted or mono- or poly-substituted; and
R₃ represents hydroxy, lower alkoxy, acyloxy, carboxy, lower alkoxycarbonyl, carbamoyl, N-mono- or N,N-di-substituted carbamoyl, amino, mono- or di-substituted amino, cycloalkyl, heterocyclyl, an aryl group, or a mono- or bi-cyclic heteroaryl group comprising 0-, 1-, 2- or 3-ring nitrogen atoms and 0 or 1 oxygen atom and 0 or 1 sulfur atom, which groups in each case are unsubstituted or mono- or poly-substituted; or
R₁ and R_{2,} together, represent alkylene with 4, 5 or 6 carbon atoms optionally mono- or di-substituted by lower alkyl, cycloalkyl, heterocyclyl, phenyl, hydroxy, lower alkoxy, amino, mono- or di-substituted amino, oxo, pyridyl, pyrazinyl or pyrimidinyl; benzalkylene with 4 or 5 carbon atoms; oxaalkylene with 1 oxygen and 3 or 4 carbon atoms; or azaalkylene with 1 nitrogen and 3 or 4 carbon atoms, wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-di-substituted carbamoyl-lower alkyl, cycloalkyl, lower alkoxycarbonyl, carboxy, phenyl, substituted phenyl, pyridinyl, pyrimidinyl or pyrazinyl;
R₄ represents hydrogen, lower alkyl or halogen;
or
(b) Py denotes 5-pyrimidyl, R₁ is hydrogen, R₂ is [[(3S)-3-(dimethylamino)- 1-pyrrolidinyl]methyl]-3-(trifluoromethyl)phenyl and R₄ is methyl;
or a pharmaceutically acceptable salt of such a compound, wherein the compound of formula I is administered once daily just before bedtime (QHS).

2. The method according to claim 1, wherein the pyrimidylaminobenzamide is 4-methyl-3-[[4-(3-pyridinyl)-2-pyrimidinyl]amino]-N-[5-(4-methyl-1H-imidazol-1-yl)-3-(trifluoromethyl)phenyl] benzamide.

3. The method according to claim 2, wherein the pyrimidylaminobenzamide is employed in the form of its hydrochloride monohydrate.

4. The method according to any one of claims 1, wherein the proliferative disorder or other pathological condition is selected from melanoma, breast cancer, cancer of the colon, lung cancer, cancer of the prostate or Kaposi's sarcoma, gastrointestinal stromal tumors (GIST), acute myeloid leukemia (AML), leukemia which responds to an inhibition of the Abl tyrosine kinase activity, mesothelioma, systemic mastocytosis, hypereosinophilic syndrome (HES), fibrosis, rheumatoid arthritis, polyarthritis, scleroderma, lupus erythematosus, graft-versus host diseases, neurofibromatosis, pulmonary hypertension, Alzheimer's disease, seminomas and dysgerminomas and psoriasis.

5. The method according to any one of claims 1, wherein the proliferative disorder is Philadelphia positive leukemia and the dose administered is between 500 mg/day and 700 mg/day.

6. A commercial package containing a pharmaceutical composition comprising a pyrimidylaminobenzamide of formula I 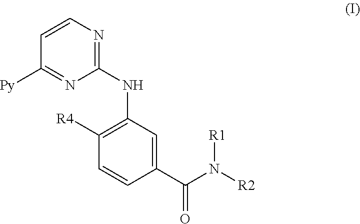
wherein
(a) Py denotes 3-pyridyl,
R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted; and
R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;
R₄ represents hydrogen, lower alkyl, or halogen;
wherein the prefix “lower” denotes a radical having up to and including a maximum of 7 carbon atoms,
or
(b) Py denotes 5-pyrimidyl, R₁ is hydrogen, R₂ is [[(3S)-3-(dimethylamino)- 1-pyrrolidinyl]methyl]-3-(trifluoromethyl)phenyl and R₄ is methyl;
or of a pharmaceutically acceptable salt thereof, respectively,
together with instructions for use for the treatment of a proliferative disorder or a pathological condition mediated by the Bcr-Abl oncoprotein, the cell transmembrane tyrosine kinase receptor c-Kit, DDR₁ (discoidin domain receptor 1), DDR₂ (discoidin domain receptor 2) or PDGF-R (platelet derived growth factor receptor) kinase activity, wherein the medicament shall be used once daily just before bedtime (QHS).
